(19) United States
(12) Patent Application Publication
(10) Pub. No.: US 2018/0353598 A1
(43) Pub. Date: Dec. 13, 2018

(54) OPTIMIZATION OF PHYSIOLOGICAL PROPERTIES OF CALCIUM PHOSPHATE AS VACCINE ADJUVANT

(71) Applicants: National Institutes of Biomedical Innovation, Health and Nutrition, Ibaraki-shi, Osaka (JP); SofSera Corporation, Shinjuku-ku (JP)

(72) Inventors: Ken Ishii, Ibaraki-shi, Osaka (JP); Masayuki Hayashi, Ibaraki-shi, Osaka (JP); Yasumichi Kogai, Shinjuku-ku, Tokyo (JP); Daisuke Nomi, Shinjuku-ku, Tokyo (JP); Karl Kazushige Kawabe, Shinjuku-ku (JP)

(21) Appl. No.: 15/761,058

(22) PCT Filed: Sep. 16, 2016
(86) PCT No.: PCT/JP2016/004250

§ 371 Date: Aug. 14, 2018

(30) Foreign Application Priority Data
Sep. 17, 2015 (JP) 2015-183631

(51) Int. Cl. A61K 39/39; A61K 9/14

(52) U.S. Cl. CPC A61K 39/39; A61K 9/14

(57) ABSTRACT

Provided is an improved biocompatible immune adjuvant. More specifically, provided are: an immune adjuvant that comprises calcium phosphate, preferably hydroxyapatite, having an average particle size of about 40 nm exclusive to about 1800 nm exclusive; a medicine that comprises this immune adjuvant; and a vaccine that comprises this immune adjuvant. Rod-shaped HAp induced inflammasome-dependent IL-1β production in vitro more strongly than spherical HAp. In WT mice, however, spherical HAp and rod-shaped HAp induced equivalent antigen responses.

### TECHNICAL FIELD

The present invention relates to an immunoadjuvant. More specifically, the present invention relates to an improvement in use of calcium phosphate such as hydroxyapatite as an immunoadjuvant.

### BACKGROUND ART

Calcium phosphate is a natural component of hard tissue in the human body, such as bones and teeth. Hydroxyapatites (HAp), which are comprised of calcium phosphate, are used in dental implant materials or in bone formation (Non Patent Literatures 1 and 2). HAp implants can be reabsorbed by either a solution mediated process (solubility of an implant in saline) (Non Patent Literature 3) or a cell-mediated process. In particular, macrophages have the ability to decompose HAp crystals (Non Patent Literature 4), demonstrating that HAp is a biodegradable material.

Vaccines are the most effective agents for preventing an infection for humans. A vaccine adjuvant is used to improve the efficacy of some vaccines. Vaccine adjuvants are classified by different types of compounds (e.g., microorganism products, emulsions, particles, or liposomes (Non Patent Literatures 5 to 7)). Calcium phosphate (CP) is a commercialized vaccine adjuvant for humans with an established safety profile (Non Patent Literature 8). CP was shown to improve the efficacy of various vaccines, including diphtheria and tetanus toxoids (Non Patent Literatures 9 and 10). CP has several advantages over other non-toxic particulate adjuvants and high adjuvant activity without inducing IgE (Non Patent Literatures 8 and 11).

A study on several different types of non-calcium phosphate particulate adjuvants has reported that the particle size, surface morphology, surface charge, and surface area are important parameters that affect adjuvant activity (Non Patent Literatures 12 and 13). Furthermore, it is reported that the inflammation promoting activity thereof significantly varies depending on the physical properties thereof. The principle therebehind is not only undiscovered, but is demonstrated to be unpredictable. Rod-shaped or needle-shaped hydroxyapatite (HAp) crystals that are similar in morphology to a hemagglutinin (HA) aggregation observed in synovial membranes in osteoarthritis (OA) induce higher production of IL-1β and IL-18 via NLRP3 inflammasome activation in a microphage than spherical hydroxyapatite crystals. This suggests that heterotopic deposition of HAp crystals in the joint induces inflammation that is closely related to OA (Non Patent Literature 14). However, the relationship thereof with adjuvant activity has not been reported. Various studies have reported that nanoparticles of HAp comprised of CP have adjuvant activity (Non Patent Literatures 11 and 15).

### CITATION LIST

### Non Patent Literature

[NPL 1] FURUHASHI., K., et al., Evaluation of Adhesion between Material and Epithelium using a Three-dimensional Human Epidermal Model. Nano Biomedicine, 2012. 4(2): p. 76-84.

[NPL 2] HATAKEYAMA., W., et al., Bone-regeneration Trial of Rat Critical-size Calvarial Defects using Nano-apatite/collagen Composites. Nano Biomedicine, 2013. 5(2): p. 95-103.

[NPL 3] JARCHO, M., Calcium Phosphate Ceramics as Hard Tissue Prosthetics

[NPL 4] Kwong C. H., et al., Solubilization of hydroxyapatite crystals by murine bone cells, macrophages and fibroblasts. Biomaterials., 1989. 10: p. 579-84.

[NPL 5] Tritto, E., F. Mosca, and E. De Gregorio, Mechanism of action of licensed vaccine adjuvants. Vaccine, 2009. 27(25-26): p. 3331-4.

[NPL 6] Brito, L. A., P. Malyala, and D. T. O'Hagan, Vaccine adjuvant formulations: a pharmaceutical perspective. Semin Immunol, 2013. 25(2): p. 130-45.

[NPL 7] Hedayat, M., K. Takeda, and N. Rezaei, Prophylactic and therapeutic implications of toll-like receptor ligands. Med Res Rev, 2012. 32(2): p. 294-325.

[NPL 8] TCHAVDAR L, V., Aluminium Phosphate but Not Calcium Phosphate Stimulates the Specific IgE Response in Guinea Pigs to Tetanus Toxoid. Allergy, 1978. 33: p. 155-9.

[NPL 9] Aggerbeck, H. and I. Heron, Adjuvanticity of aluminium hydroxide and calcium phosphate in diphtheria-tetanus vaccines-I. Vaccine, 1995. 13(14): p. 1360-5.

[NPL 10] Aggerbeck, H., C. Fenger, and I. Heron, Booster vaccination against diphtheria and tetanus in man. Comparison of calcium phosphate and aluminium hydroxide as adjuvants-II. Vaccine, 1995. 13(14): p. 1366-74.

[NPL 11] HE, Q., et al., Calcium Phosphate Nanoparticle Adjuvant. CLINICAL AND DIAGNOSTIC LABORATORY IMMUNOLOGY, 2000. 7(6): p. 899.

[NPL 12] Kuroda, E., C. Coban, and K. J. Ishii, Particulate Adjuvant and Innate Immunity: Past Achievements, Present Findings, and Future Prospects. International Reviews of Immunology, 2013. 32(2): p. 209-220.

[NPL 13] Jiang, D., et al., Structure and adsorption properties of commercial calcium phosphate adjuvant. Vaccine, 2004. 23(5): p. 693-8.

[NPL 14] Jin, C., et al., NLRP3 inflammasome plays a critical role in the pathogenesis of hydroxyapatite-associated arthropathy. Proc Natl Acad Sci USA, 2011. 108(36): p. 14867-72.

[NPL 15] He, Q., et al., Calcium Phosphate Nanoparticles Induce Mucosal Immunity and Protection against Herpes Simplex Virus Type 2. Clinical and Vaccine Immunology, 2002. 9(5): p. 1021-1024.

### SUMMARY OF INVENTION

### Solution to Problem

The inventors have tested the role of size or shape of HAp in an antibody response after immunization with an antigen to discover that HAp with a diameter (average particle size) greater than about 40 nm and less than about 1800 nm, preferably in the range of about 100 to about 400 nm, induces a significantly higher antibody response compared to smaller or larger HAp to complete the present invention. The optimal size or shape for adjuvant activity is unknown, and prediction or estimation thereof is impossible. In view of such a state of the art, it was unexpected that adjuvant activity is extremely high at such a specific size. In particular, about 40 nm induced the strongest ovalbumin (OVA) specific antibody response in a comparison of immune responses induced by OVA conjugated polystyrene beads of different sizes (about 20 nm, about 40 nm, about 100 nm, about 500 nm, about 1 μm, and about 2 μm) (Fifis, T., et al., Size-Dependent Immunogenicity: Therapeutic and Protective Properties of Nano-Vaccines against Tumors. The Journal of Immunology, 2004. 173(5): p. 3148-3154). In view of the above, this is a result that is rather unexpected from information that is currently available.

In the present invention, rod-shaped HAp induced stronger inflammasome dependent IL-1β production in vitro than spherical HAp in a comparison between spherical and rod-shaped HAp. However, spherical and rod-shaped HAp induced equivalent antibody responses in WT mice. Similarly, an antibody response at a level equivalent to HAp adjuvanted vaccine inoculation was induced in Nlrp3−/− mice, Asc−/− mice, and Caspase1−/− mice. In summary, the results of the inventors demonstrated that the size is a more important property than shape, and production of IL-1β via NLRP3 inflammasomes is not required for adjuvant activity of HAp in mice.

Thus, the present invention provides the following.

(1) An immunoadjuvant comprising calcium phosphate with an average particle size greater than about 40 nm and less than about 1800 nm.
(2) The immunoadjuvant of item 1, wherein the average particle size of the calcium phosphate is about 100 to about 400 nm.
(3) The immunoadjuvant of item 1 or 2, wherein the calcium phosphate is a hydroxyapatite.
(4) The immunoadjuvant of any one of items 1 to 3, wherein the calcium phosphate is rod-shaped.
(5) An immunoadjuvant comprising calcium phosphate, wherein the calcium phosphate is rod-shaped.
(6) The immunoadjuvant of any one of items 1 to 5 for use as an immunoadjuvant, which does not require NLRP3 inflammasome activation.
(7) The immunoadjuvant of any one of items 1 to 6, wherein the immunoadjuvant enhances a Th2 response.
(8) A medicament comprising the immunoadjuvant of any one of items 1 to 7.
(9) A vaccine comprising the immunoadjuvant of any one of items 1 to 7.
(10) Calcium phosphate with an average particle size greater than about 40 nm and less than about 1800 nm for use as an immunoadjuvant.
(11) Rod-shaped calcium phosphate for use as an immunoadjuvant.
(11A) Calcium phosphate of item 10 or 11, comprising one or more features of items 1 to 7.
(12) A method of preventing or treating a disease requiring a vaccine comprising an immunoadjuvant comprising calcium phosphate with an average particle size greater than about 40 nm and less than about 1800 nm, comprising administering to a subject an effective amount of the vaccine.
(13) A method of preventing or treating a disease requiring a vaccine comprising an immunoadjuvant comprising rod-shaped calcium phosphate, comprising administering to a subject an effective amount of the vaccine.
(13A) The method of item 12 or 13, comprising one or more of the features of items 1 to 7.

In the present invention, one or more of the features disclosed above are intended to be provided not only as the explicitly disclosed combinations, but also as other combinations thereof. Additional embodiments and advantages of the present invention are recognized by those skilled in the art by reading and understanding the following detailed description, as needed.

### Advantageous Effects of Invention

The particles of the present invention induced a high antibody response (e.g., FIGS. 2A to 2C). Although not wishing to be bound by any theory, immunoglobulin G (IgG) subclass analysis has revealed that HAp induced greater production of IgG1 than IgG2c. This has led to the discovery that HAp has a tendency to induce a Th2 polarized immune response. In addition, it was demonstrated that a high virus neutralization titer was exhibited from immunization with inactivated split vaccines (SV)+about 100 nm to about 400 nm hydroxyapatite (HAp). Thus, the present invention demonstrates that neutralization capability is also further enhanced by using particles within this specific range.

### BRIEF DESCRIPTION OF DRAWINGS

FIG. 1 shows spherical hydroxyapatites (HAp) of various sizes. FIG. 1 shows pictures of (A) S40, (B) S100, (C) S170, (D) S400, (E) S1800, and (F) S5000 from a scanning electron microscope (SEM). See Table 1 for each symbol. The pictures of the left side are at low magnification and the pictures on the right side are enlarged SEM pictures of HAp at high magnification. The pictures on the left and right were taken at a magnification of (A to D) 10,000 or 40,000, (E) 2,000 or 40,000, and (F) 1,000 or 10,000.

FIGS. 2-1 and 2-2 show that several hundred nanometer HAp induces a high antibody response. On day 0 and day 14, mice (n=5) were immunized by 1 μg of inactivated split vaccine (SV) alone or i.d. with an adjuvant. The antigen specific (A) total IgG, (B) IgG1, and (C) IgG2c titers in the serum on day 14 (white) and day 28 (black) were measured by ELISA. The vertical axes of A to C indicate the anti-SV titer. (D) The virus neutralization titer in the serum on day 28 was measured by microneutralization assay. The results show a representative of three independent experiments. Alum indicates alum. Each particle of S40, S100, S170, S400, S1800, and S5000 has the shape disclosed in Table (S means sphere). The vertical axis of D indicates the neutralization titer. The numerical values on the horizontal axes of A to D indicate the amount used (mg). The median value and SEM are shown for each group. Statistical significance is shown as *P<0.05, **P<0.01, and ***P<0.001 in Dunnett's multiple comparison test.

FIGS. 2-1 and 2-2 show that several hundred nanometer HAp induces a high antibody response. On day 0 and day 14, mice (n=5) were immunized by 1 μg of inactivated split vaccine (SV) alone or i.d. with an adjuvant. The antigen specific (A) total IgG, (B) IgG1, and (C) IgG2c titers in the serum on day 14 (white) and day 28 (black) were measured by ELISA. The vertical axes of A to C indicate the anti-SV titer. (D) The virus neutralization titer in the serum on day 28 was measured by microneutralization assay. The results show a representative of three independent experiments. Alum indicates alum. Each particle of S40, S100, S170, S400, S1800, and S5000 has the shape disclosed in Table 1 (S means sphere). The vertical axis of D indicates the neutralization titer. The numerical values on the horizontal axes of A to D indicate the amount used (mg). The median value and SEM are shown for each group. Statistical significance is shown as *P<0.05, **P<0.01, and ***P<0.001 in Dunnett's multiple comparison test.

FIG. 3 shows rod-shaped HAp of various sizes. Pictures from a scanning electron microscope for (A) R120, (B) R160, and (C) R250 are shown (R means rod). See Table 1 for each symbol. The pictures on the left and right were taken at a magnification of 10,000 and 40,000, respectively. Each bar refers to the corresponding length.

FIGS. 4-1 and 4-2 show that rod-shaped HAp also has adjuvant activity. On day 0 and day 14, mice (n=5) were immunized by 1 μg of SV alone or i.d. with an adjuvant. The antigen specific (A) total IgG, (B) IgG1, and (C) IgG2 titers in the serum on day 14 (white) and day 28 (black) were measured by ELISA. The vertical axes indicate the anti-SV titer. (D) The virus neutralization titer in the serum on day was measured by microneutralization assay. The results show a representative of three independent experiments. See Table 1 for each of R120, R160, and R250. For D, the vertical axis indicates the neutralization titer. The numerical values on the horizontal axes of A to D indicate the amount used (mg). The median value and SEM are shown for each group. Statistical significance is shown as *P<0.05, **P<0.01, and ***P<0.001 in Student Dunnett multiple comparison test.

FIGS. 4-1 and 4-2 show that rod-shaped HAp also has adjuvant activity. On day 0 and day 14, mice (n=5) were immunized by 1 μg of SV alone or i.d. with an adjuvant. The antigen specific (A) total IgG, (B) IgG1, and (C) IgG2 titers in the serum on day 14 (white) and day 28 (black) were measured by ELISA. The vertical axes indicate the anti-SV titer. (D) The virus neutralization titer in the serum on day was measured by microneutralization assay. The results show a representative of three independent experiments. See Table 1 for each of R120, R160, and R250. For D, the vertical axis indicates the neutralization titer. The numerical values on the horizontal axes of A to D indicate the amount used (mg). The median value and SEM are shown for each group. Statistical significance is shown as *P<0.05, **P<0.01, and ***P<0.001 in Student Dunnett multiple comparison test.

FIG. 5 shows that several hundred nanometer HAp induces IL-113 production via an inflammasome in a macrophage in vitro. Peritoneal macrophages prepared from (A) C57BL/6j mice or (B) Caspase1−/− mice were primed for 15 hours with a ng/mL LPS (white indicates no lipopolysaccharide (LPS), and black indicates LPS), and were then stimulated with HAp or alum for 8 hours at the indicated concentration. IL-1β secreted to the culture supernatant was measured by ELISA. The results show a representative of three independent experiments. The vertical axis indicates the concentration of IL-1β, and the horizontal axis indicates the amount used (mg). The four bars on the left indicate spherical HAp. Third to fifth bars indicate rod-shaped HAp. Second from the right bar indicates alum. The right end indicates none. The median value and SEM are shown for each group. Statistical significance compared to an unstimulated group is shown as ***P<0.001 in Dunnett's multiple comparison test.

FIG. 6 shows that NLRP3 inflammasomes activation is not required for adjuvant activity of HAp. On day 0 and day 14, (A) Nlrp3−/− mice, (B) Asc−/− mice or (C) Caspase1−/− mice (n=3 to 6) were immunized by 10 μg of OVA alone or i.d. with an adjuvant. 5 mg of 5170 and R160 was used per mouse. The antigen specific total IgG titers in the serum on day 14 (white) and day 28 (black) were measured by ELISA. The vertical axis indicates the anti-OVA titer. 5170 and R160 each have the shape disclosed in Table 1. Alum indicates alum. The results show a representative of two independent experiments. The median value and SEM are shown for each group. Statistical significance was determined by Student's t-test.

FIG. 7 shows that peritoneal macrophages prepared from (A) C57BL/6j mice or (B) Caspase1−/− mice were primed for 15 hours with 50 ng/mL of LPS, and were then stimulated with HAp or alum for 8 hours at the indicated concentration. Tumor necrosis factor α (TNFα) secreted in the culture supernatant was measured by ELISA. The results show a representative of three independent experiments. The vertical axis indicates the concentration of TNFα, and the horizontal axis indicates the amount used (mg). The four bars on the left indicate spherical HAp. Third to fifth bars indicate rod-shaped HAp. Second from the right bar indicates alum. The right end indicates none. The median value and SEM are shown for each group. Statistical significance compared to an unstimulated group was determined by a Dunnett's multiple comparison test.

### DESCRIPTION OF EMBODIMENTS

The present invention is explained hereinafter. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

As used herein, “calcium phosphate” specifically includes hydroxyapatite (Ca₁₀(PO₄)₆ (OH)₂), tricalcium phosphate (Ca₃(PO₄)₂), calcium metaphosphate (Ca(PO₃)₂), fluorapatite (Ca₁₀(PO₄)₆F₂), chlorapatite (Ca₁₀(PO₄)₆Cl₂), and the like. Such calcium phosphate may be used alone or as a combination of two or more. Among such calcium phosphate, hydroxyapatites are preferably used from the viewpoint of the excellent adjuvant property, adsorbency, biocompatibility, retention and growth of implanted cells, and the like, but the present invention is not limited thereto.

Inorganic compounds such as the above calcium phosphate are preferably sintered substances subjected to sintering. Use of a sintered substance of the above inorganic compounds can impart heat resistance or chemical stability.

Hydroxyapatite (HAp) is a basic calcium phosphate represented by the chemical formula Ca₁₀(PO₄)₆(OH)₂, which is known to be present in natural as an ore or as the main component of bones and teeth, and to exhibit high biocompatibility. Hydroxyapatites are synthesized by various methods and utilized in various fields, including biomaterials. The manufacturing methods thereof include a solution method (wet process), which is a method of synthesis by reacting a calcium ion and a phosphate ion in a neutral or alkaline aqueous solution at room temperature. Representative methods include those using a neutralization reaction and those using a reaction of a salt and a salt. HAp synthesized by the above approaches is amorphous, so that sufficient stability cannot be guaranteed depending on the intended use. Thus, it can be preferable to further sinter HAp to enhance the crystallinity.

Hydroxyapatites may be spherical or rod-shaped. Examples of such products include SHAp (calcined hydroxyapatite nanoparticles) sold by SofSera, which are sold as, for example, spherical or rod shaped particles.

As used herein, “average particle size” is the average diameter of particles when referring to the calcium phosphate or immunoadjuvant of the present invention. Numerical values measured as follows are used herein as an average particle size. As used herein, “diameter” refers to “average particle size”, unless specifically noted otherwise. A scanning electron microscope (SEM) is used for measuring an average particle size herein. A detailed procedure of calculating an average particle size is the following.

*An SEM is used to capture target hydroxyapatite particles at a 40,000× magnification in 9 fields of vision.
*The lengths of 12 hydroxyapatite particles per image for a total of 108 hydroxyapatite particles are measured in the captured images.
*The arithmetic mean of the obtained 108 lengths is calculated as an average particle size.

As used herein, “spherical” and “substantially spherical” are interchangeably used. For hydroxyapatites, those with a ratio of the shortest diameter to the longest diameter of target particles of less than 2 are referred to thereby. “Spherical” and “substantially spherical” include completely spherical as well as shapes that are somewhat non-spherical. Plus and minus “spherical” and “substantially spherical” hydroxyapatites generally coexist. They are also called “substantially spherical” in the art, but “spherical” and “substantially spherical” are used synonymously for the present invention.

As used herein, “rod-shaped” (rod-like=rod) refers to so-called stick-like hydroxyapatites in addition to non-spherical hydroxyapatites when used for hydroxyapatites, referring to those with a ratio of the shortest diameter to the longest diameter of target particles of about 2 or greater. “Rod-shaped” hydroxyapatites are generally divided into plus (side surface) and minus (cross-section). Although not wishing to be bound by any theory, it is explained that a rod-shaped cross-section (c face) exposes many oxygen atoms from phosphate ions, and a rod-shaped side surface (a face) exposes many calcium atoms, and charges from each atom (ion) results in a difference in the distribution of plus/minus in rod-shapes and spheres (KAWASAKI et al., European Journal of Biochemistry 152, 361-371 (1985)). Although not wishing to be bound by any theory, while nanoparticles are not completely dispersed because they are nanoparticles, the zeta potential tends to exhibit a value that is more towards plus for rod-shape compared to spheres. Since this can be explained by the ratio of areas and localization of charges exposed on the surface, the tendency of ±can also be explained.

As used herein, “(immuno)adjuvant” is a term derived from the word “adjuvare”, which means “assist” in Latin. “(Immuno)adjuvant” is a general term for substances (agents) administered with a vaccine and used for enhancing an effect thereof (immunogenicity). It has been revealed that activation of innate immunity by an adjuvant is essential for acquiring an effective immunity (vaccine effect). Adjuvants can enhance any immune response, but may enhance a response polarized to one of cellular immunity (Th1 immunity) and humoral immunity (Th2 immunity) or both.

(Manufacture and Particle Size Modulation of Calcium Phosphate Particles)

Commercially available particles can be used as the particles of the present invention, but particles of the present invention can be produced by the following step of manufacturing nanoparticles (see for example Japanese Laid-Open Publication No. 2010-235686). A nanoparticle manufacturing step is disclosed below while using hydroxyapatite particles which are one example of nanoparticles as an example, but the nanoparticle manufacturing step is not limited thereto.

The aforementioned nanoparticle manufacturing step preferably comprises a “primary particle generating step” and/or “sintering step”. It is more preferable to comprise a “removing step” and/or “mixing step” in addition to the aforementioned steps. It is preferable that the aforementioned four steps are performed in the order of “A. primary particle generating step”→“B. mixing step”→“C. sintering step”→“D. removing step”.

A nanoparticle manufacturing step comprising all four of the aforementioned steps is disclosed below, but the manufacturing step is not limited thereto.

(A. Primary Particle Generating Step)

As used herein, “primary particle” refers to a particle formed using calcium phosphate such as hydroxyapatite (HAp) before a sintering step. In other words, a primary particle refers to a particle that is initially formed in a manufacturing step of hydroxyapatite particles. A primary particle, when narrowly defined, refers to a monocrystalline particle. As used herein, “primary particle” includes amorphous particles and sintered particles that have been subsequently sintered.

Meanwhile, “secondary particle” refers to a particle formed of multiple “primary particles” bound to one another by a physical bond (e.g., fusion) or chemical bond (e.g., ionic bond, covalent bond, or the like). The number of primary particles that bind to one another, the shape after binding, and the like upon forming a secondary particle are not particularly limited.

In particular, “monocrystalline primary particle” refers to a primary particle consisting of a monocrystal of calcium phosphate such as hydroxyapatite or a mass of such primary particles consisting of monocrystals, which has aggregated due to an ionic interaction. A “mass of particles, which has aggregated due to an ionic interaction” is a mass of particles that self-aggregate by an ionic interaction when dispersed in a medium comprising water or an organic solvent. A secondary particle, which is formed when particles melt and are polycrystallized due to sintering, is not encompassed.

A primary particle generating step is not particularly limited and may be any step capable of generating the aforementioned primary particle. For example, it is sufficient to gradually add an aqueous (NH₄)₂HPO₄ solution with a pH adjusted to an alkaline pH (e.g., pH of 12.0) to an aqueous Ca(NO₃)₂ solution with a pH adjusted to an alkaline pH (e.g., pH of 12.0) at a high temperature (e.g., 80° C.).

The state (e.g., particle size and particle size distribution) of primary particles generated by a primary particle generating step is reflected in the state (e.g., particle size and particle size distribution) of calcium phosphate such as hydroxyapatites. Thus, if it is desirable to manufacture calcium phosphate particles such as hydroxyapatites with a small particle size (nano size) and homogeneous particle size (narrow particle size distribution), it is preferable to generate primary particles with a small particle size (nano size) and homogeneous particle size (narrow particle size distribution) in the primary particle generating step.

While the particle size of primary particles is not particularly limited, it is preferable that the average particle size is about 10 nm to about 1000 nm, more preferable about 10 nm to about 700 nm, and still preferably about 20 nm to about 600 nm, still more preferably about 25 nm to about 500 nm, or has the ultimately desired average particle size (e.g., between about 100 nm and about 400 nm) from the beginning. Of course, if the average particle diameter can be changed to the ultimately desired average particle size (e.g., between about 100 nm and about 400 nm), this is not limited thereto. The coefficient of variation of particle size of a group of primary particles consisting of primary particles is preferably about 20% or less, more preferably about 18% or less, and most preferably about 15% or less. It is sufficient to use dynamic light scattering or an electron microscope to measure particle sizes of at least about 100 primary particles to calculate the coefficient of variation and particle size of primary particles based on a result of such a measurement. A “coefficient of variation” is a value indicating the variation in particle sizes of particles, which can be calculated by standard deviation/average particle size×100(%).

A method of making primary particles with a small particle size (nano size) and homogeneous particle size (narrow particle size distribution) disclosed above is not particularly limited. For example, a method disclosed in Japanese Laid-Open Publication No. 2002-137910 can be utilized. In other words, primary particles can also be synthesized by solubilizing a calcium solution and a phosphate solution and mixing and reacting them with a surfactant/water/oil based emulsion phase at or above the cloud point of the surfactant. The size of primary particles can also be controlled by changing the hydrophilic/hydrophobic ratio and a functional group of the aforementioned surfactant during synthesis. However, a method of making primary particles is not limited thereto.

The following is a brief explanation of the principle of manufacturing primary particles disclosed above. In a method of synthesizing hydroxyapatite microparticles by mixing a calcium solution and a phosphate solution with a surfactant/water/oil based emulsion phase, a nucleus of a hydroxyapatite grows in a micelle of a surfactant and grows a crystal. The thermodynamic stability of the micelle can be modulated by setting the reaction temperature at this point to or above the cloud point of the surfactant. In other words, the ability to form a micelle of a surfactant can be reduced by raising the reaction temperature to or above the cloud point of the surfactant. As a result, the driving force for the crystal growth of hydroxyapatites, which has been restricted within the framework of a micelle, is greater than the driving force for attempting to maintain the framework of the micelle. This mechanism can be utilized to control the morphology (e.g., shape, size, or the like) of a crystal.

When a micelle is made with a surfactant, a functional group of the surfactant (hydrophilic site) and the hydrophilic/hydrophobic ratio of the surfactant are important. A difference therein results in a different micelle stability and cloud point. The cloud point of a surfactant varies depending on the type of surfactant. Thus, the micelle stability and cloud point can be changed by appropriately changing the type of surfactant. The size of hydroxyapatite microparticles can be controlled thereby.

### Disclosure of Preferred Embodiments

The preferred embodiments of the present invention are disclosed hereinafter. It is understood that the embodiments provided hereinafter are provided to better facilitate the understanding of the present invention, so that the scope of the present invention should not be limited by the following description. Thus, it is apparent that those skilled in the art can refer to the descriptions herein to appropriately make modifications within the scope of the present invention. It is also understood that the following embodiments of the present invention can be used individually or as a combination.

(Use of Hydroxyapatite as a Vaccine Adjuvant)

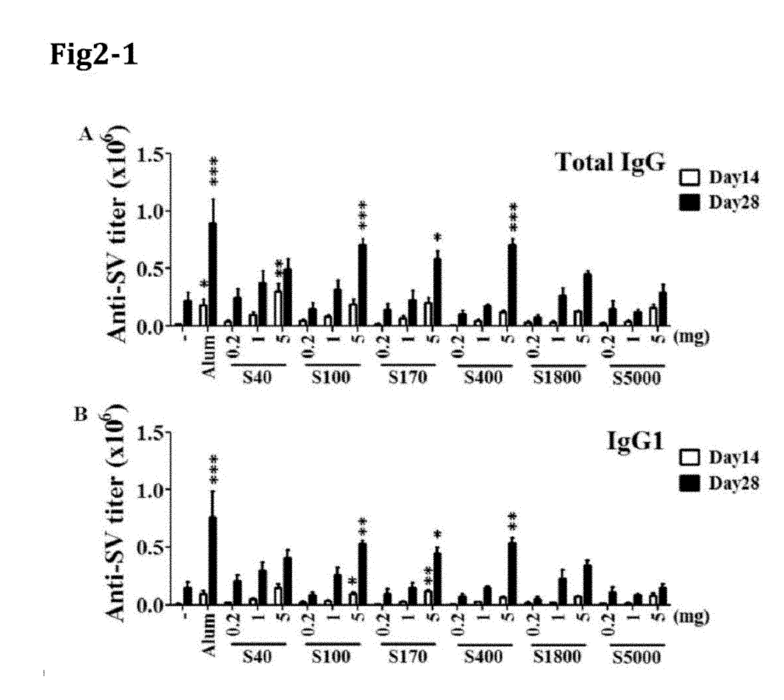

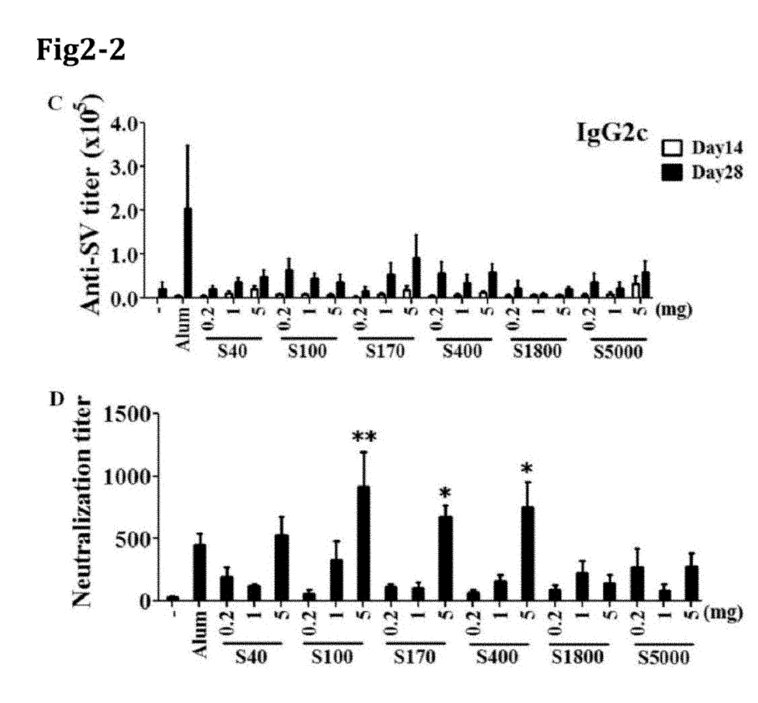

In one aspect, the present invention provides an immunoadjuvant comprising calcium phosphate with an average particle size greater than about 40 nm and less than about 1800 nm. Although not wishing to be bound by any theory, this is because it was discovered that calcium phosphate with a particle size greater than about 40 nm and less than about 1800 nm, such as particles of about 100 to about 400 nm, induces a higher antibody response than particles that are smaller (about 40 nm) and larger (about 1.8 μm or about 5 μm) (e.g., FIGS. 2A to 2C). Although not wishing to be bound by any theory, IgG subclass analysis has revealed that hydroxyapatites (HAp) induced greater production of IgG1 than IgG2c, which has a tendency to result in Th2 polarized immune responses, but the present invention is not limited thereto. In addition, it is demonstrated that immunization using an inactivated split vaccine (SV) with about 100 to about 400 nm hydroxyapatite (HAp) particles exhibits a higher virus neutralization titer than immunization using a particle size than is smaller (about 40 nm) and larger (about 1.8 μm or about 5 μm). Neutralizing capability is also demonstrated to be enhanced by using particles in this specific range.

Various particles of alum, silica, and the like are known to act as an adjuvant when co-administered with a vaccine antigen. In addition, it is reported that the adjuvant activity is strongly affected by physicochemical properties of a particle such as the size, shape and surface charge. However, there was no progress in the elucidation of the relationship between the required property and adjuvant activity thereof. Thus, demonstration of the hydroxyapatite particles (HAp) comprised of calcium phosphate of the present invention functioning as an adjuvant in mice at a specific size is noteworthy as a surprising result.

In the embodiments of the present invention, the lower limit of the diameter of calcium phosphate used in the immunoadjuvant of the present invention may be about 40 nm, about 45 nm, about 50 nm, about 55 nm, about 60 nm, about 65 nm, about 70 nm, about 75 nm, about 80 nm, about 85 nm, about 90 nm, about 95 nm, or about 100 nm, and the upper limit of the diameter of calcium phosphate used in the immunoadjuvant of the present invention may be about 1800 nm, about 1700 nm, about 1600 nm, about 1500 nm, about 1400 nm, about 1300 nm, about 1200 nm, about 1100 nm, about 1000 nm, about 950 nm, about 900 nm, about 850 nm, about 800 nm, about 750 nm, about 700 nm, about 650 nm, about 600 nm, about 550 nm, about 500 nm, about 450 nm, or about 400 nm. A range of any combination of such upper limits and lower limits is intended in the present invention.

In a preferred embodiment, the diameter of calcium phosphate used in the immunoadjuvant of the present invention is about 100 to about 400 nm. More specifically, a diameter of about 100 to about 400 nm, more preferably about 100 to about 250 nm, about 120 to about 250 nm, about 120 to about 170 nm, about 100 to about 170 nm, or about 160 to about 170 nm may be advantageous in a preferred embodiment. For example, a rod-shaped calcium phosphate of about 120 to about 250 nm may be advantageous.

In a preferred embodiment, calcium phosphate used in the immunoadjuvant of the present invention is a hydroxyapatite. More preferably, the calcium phosphate used in the immunoadjuvant of the present invention is a hydroxyapatite with a diameter of about 100 to about 400 nm.

Modulation of such a diameter of hydroxyapatites can be materialized using the approach disclosed herein or another known approach.

In one preferred embodiment, calcium phosphate used in the immunoadjuvant of the present invention is rod-shaped. Although not wishing to be bound by any theory, this is because it was discovered that rod-shaped HAp induces higher interleukin 1β (IL-1β) production than spherical HAp.

In one preferred embodiment, the immunoadjuvant of the present invention is used as an immunoadjuvant, which does not require NLRP3 (NOD-like receptor family, pyrin domain containing 3) inflammasome activation.

In a preferred embodiment, the immunoadjuvant of the present invention is used as an immunoadjuvant for inducing or enhancing a Th2 (polarized) immune response. In another embodiment, the immunoadjuvant of the present invention is used as an immunoadjuvant for inducing or enhancing a Th2 (polarized) immune response using a hydroxyapatite as calcium phosphate. Th2 immunity involves humoral immunity, whereby antibody production is increased.

In one aspect, the present invention provides a medicament comprising the immunoadjuvant of the present invention. Any component for which enhancement in immunogenicity is required or desirable is contained as a component contained with the immunoadjuvant of the present invention. Typical examples of such a medicament include, but are not limited to, vaccines such as live attenuated vaccines (LAIV), inactivated whole vaccines (WV), inactivated split vaccines (SV), subunit vaccines (SU), toxoid vaccines, virus-like particles (VLP), and the like.

Examples of targets of vaccined that can be used in the present invention include, but are not limited to, influenza virus, Japanese encephalitis, diphtheria/pertussis/tetanus/polio mixture (DPTP), diphtheria/pertussis/tetanus mixture (DPT), diphtheria/tetanus mixture (DT), measles, rubella, polio, BCG, hepatitis A, hepatitis B, rabies, tetanus toxoid, diphtheria toxoid, pneumococcus, Weil's disease autumnalis, chicken pox, mumps, yellow fever, rotavirus, Haemophilus influenzae b, human papilloma virus (HPV), RS virus (RSV), cytomegalovirus (CMV), norovirus, herpes simplex virus, and the like.

Examples of subjects of vaccined that can be used in the present invention include any mammal, e.g., rodents such as mice, rats, hamsters, and Guinea pigs, Lagomorpha such as rabbits, ungulates such as pigs, cows, goats, horses, and sheep, dogs, carnivore such as cats, primates such as humans, monkeys, rhesus monkeys, cynomolgus monkeys, marmosets, orangutans, and chimpanzees, and the like. Mammals are preferably rodents (mice or the like) or primates (humans or the like), more preferably primates, and still more preferably humans.

The medicament or vaccine of the present invention can be administered by an injection via an intravenous route, peritoneal route, subcutaneous route, intradermal route, route in fat tissue, route in mammary tissue, or intramuscular route; gas induced particle bombardment (with an electron gun or the like); method through a mucosal route in a form such as nasal spray or the like; and the like. For example, the medicament or vaccine of the present invention is subcutaneously or intramuscularly injected, preferably subcutaneously injected.

In another embodiment, the medicament or vaccine of the present invention is subcutaneously administered by a needleless syringe. A needleless syringe is preferably a pressure syringe. Examples of needleless syringes include, but are not limited to, ShimaJET (Shimadzu Corporation), Twin-Jector EZ II (Nihon Chemical Research), Syrijet (Keystone), ZENEO (Crossject), and the like.

To induce an excellent immune response, the medicament or vaccine of the present invention is preferably administered multiple times at a certain interval. The number of administration can be appropriately determined while monitoring the strength of an immune response. Generally, the number of administrations may be a single administration, or 2 to 10 times, preferably 2 to 6 times, more preferably 2 to 4 times, and most preferably 3 times, but the number is not limited thereto.

The frequency of administration is generally once every 3 days to 3 months, preferably once every 1 to 4 weeks, more preferably once every 1.5 to 3 weeks, and most preferably once every 2 weeks. For example, the medicament or vaccine of the present invention may be administered three times in a two week interval to a target mammal.

Thus, the present invention also provides a method of preventing or treating a target disease by using the medicament of vaccine of the present invention.

As used herein, “X to Y” indicating a range refers to “X or greater and Y or less”. Further, “about” indicates the significant figure thereof, unless specifically noted otherwise. Since 15% tolerance is accepted for a measurement value of a diameter, about 100 nm indicates a range of 85 nm to 115 nm. Thus, it is understood that 100 nm indicates a range of 85 nm to 115 nm even without “about”, because acceptance of 15% tolerance in measurement values is presumed.

Reference literatures such as scientific literatures, patents, and patent applications cited herein are incorporated herein by reference to the same extent that the entirety of each document is specifically described.

The present invention has been disclosed while showing preferred embodiments to facilitate understanding. The present invention is disclosed hereinafter based on Examples. The aforementioned description and the following Examples are not provided to limit the present invention, but for the sole purpose of exemplification. Thus, the scope of the present invention is not limited to the embodiments and Examples that are specifically disclosed herein and is limited only by the scope of claims.

### EXAMPLES

In the Examples, the inventors provide examples demonstrating the development of a unique method of synthesizing hydroxyapatite particles (hereinafter, abbreviated as “HAp” herein in some cases) whose size and shape are desirably modulated. The inventors have tested the relationship between required physicochemical properties and adjuvant activity of HAp in mice by using HAp with several different sizes and shapes. The products disclosed in the Examples were specifically used as the reagents, but the products can also be substituted with equivalent products from other manufacturers (Sigma, Wako Pure Chemical, Nacalai Tesque, or the like).

(Materials and Methods)

(Mouse)

6-week old female C57BL/6j mice were purchased from CLEA Japan. Nlrp3−/− mice, Asc−/− mice, and Caspase1−/− mice have been previously described (Onishi, M., et al., Hydroxypropyl-beta-Cyclodextrin Spikes Local Inflammation That Induces Th2 Cell and T Follicular Helper Cell Responses to the Coadministered Antigen. J Immunol, 2015.) All animal experiments were conducted in accordance with the institutional guidelines for the animal facility of the National Institutes of Biomedical Innovation, Health and Nutrition.

(Antigen, Antibody, Adjuvant, and Peptide)

Ovalbumin (OVA) was purchased from Seikagaku Corporation (Japan). Split HA vaccines from the A/California/7/2009 (H1N1) strain was provided by the Institute of Microbial Chemistry (Japan, Osaka). Aluminum hydroxide (alum) was purchased from Invivogen. A cytokine ELISA kit for IL-1β and TNFα was purchased from R&D systems.

(Hydroxyapatite Particles (HAp))

A preparation method of HAp has been previously described (Okada, M. and T. Furuzono, Nano-Sized Ceramic Particles of Hydroxyapatite Calcined with an Anti-Sintering Agent. Journal of Nanoscience and Nanotechnology, 2007. 7(3): p. 848-851., Okada, M. and T. Furuzono, Calcination of Rod-like Hydroxyapatite Nanocrystals with an Anti-sintering Agent Surrounding the Crystals. Journal of Nanoparticle Research, 2006. 9(5): p. 807-815.), which was partially modified. Briefly, HAp synthesized by a previously method was sterilized by 2 hours of dry heating at 300° C. in a pre-sterilization glass ampule. After sealing the ampule, dry heating was repeated.

Commercially available hydroxyapatites from SofSera (Tokyo, Japan) can be used. Hydroxyapatites can also be manufactured by the following manufacturing example in addition to the aforementioned manufacturing method.

### Manufacturing Example

1. Preparation of Hydroxyapatite Particles with Low Crystallinity

Hydroxyapatite particles with low crystallinity in a spherical form were prepared by the following wet process. Ca(NO₃)₂.4H₂O and (NH₄)₂HPO₄ manufactured by Nacalai Tesque were used. 25% ammonia water manufactured by Wako Pure Chemical Industries, Ltd was used. Milli-Q water was used as pure water.

First, aqueous Ca(NO₃)₂ solution (42 mN, 80 mL) adjusted to a pH of 12 with 25% ammonium water was poured into a 1 L flask, to which a cooling tube and a crescent mixer blade were connected, and was maintained at room temperature. An aqueous (NH₄)₂HPO₄ solution (100 mN, 200 mL) adjusted to a pH of 12 with ammonium water was added to the flask at room temperature. The mixture was reacted for 10 hours. The resulting reactant was then separated and washed by centrifugation to obtain hydroxyapatite particles with low crystallinity.

2. Preparation of Hydroxyapatite Particles with High Crystallinity

The hydroxyapatite particles with low crystallinity obtained above were baked by the following method to make hydroxyapatite particles with high crystallinity.

First, 0.5 g of hydroxyapatite particles with low crystallinity was dispersed in 100 mL of an aqueous solution with a pH of 7.0 comprising 0.5 g of polyacrylic acid (ALDRICH, weight-average molecular weight of 15,000 g/mol) as a fusion preventing agent (hereinafter, aqueous solution A) to allow polyacrylic acid to adsorb onto the particle surface. 500 mL of saturated aqueous calcium hydroxide [Ca(OH)₂] solution was then added to the dispersion prepared above to have calcium polyacrylate precipitate on the particle surface. Precipitates generated as a result were collected and dried at 80° C. under reduced pressure to obtain mixed particles.

The aforementioned mixed particles were placed in a crucible for 1 hour of sintering at a sintering temperature of 800° C. Calcium polyacrylate was thermally decomposed at this time to be calcium oxide [CaO].

The resulting sintered compact was then suspended into 500 mL of aqueous solution A prepared above, separated and washed by centrifugation, further suspended in distilled water, and similarly separated and washed by centrifugation to remove the fusion preventing agent and ammonium nitrate and collect hydroxyapatite particles with high crystallinity.

When the resulting hydroxyapatite particles with high crystallinity were observed with a scanning electron microscope to measure the average particle size, the average particle size was 48 nm. The scanning electron microscope manufactured by JEOL Ltd. with a model number JSM-6301F was used for said observation at 90,000 times magnification.

(Scanning Electron Microscope (SEM) Analysis)

The size and shape of HAp were observed with an SEM (JSM-6301F; JEOL Ltd., Tokyo, Japan).

(Immunization)

C57BL/6J was immunized i.d. (base of tail) twice at a 2 week interval (day 0 and day 14). For antigen specific ELISA, blood samples were collected on day 14 and day 28. Mice were anesthetized with ketamine during vaccination and bleeding. Antigen loaded alum or HAp was rotated for 1 hour or longer prior to immunization. 0.67 mg of alum was used for each mouse for immunization.

(Ab Titer)

A 96-well plate was coated with 1 μg/mL of split HA for a split HA vaccination group and WV vaccination group, with 10 μg/mL of OVA for an OVA vaccination group, and with 1 μg/mL of HB for an HB vaccination group in a carbonate buffer (pH of 9.6) for ELISA. The well was blocked with 1% BSA containing phosphate buffered saline (PBS). Diluted serum from an immunized mouse was incubated on a plate that was coated with an antigen. After washing, goat anti-mouse total IgG conjugated horseradish peroxidase (HRP), goat anti-mouse IgG1 conjugated HRP, or goat anti-mouse IgG2c conjugated HRP (Southern Biotech) was added and incubated at room temperature for 1 hour. After further washing, the plate was incubated for 30 minutes with 3,3′,5,5′-tetramethylbenzidine (TMB). The reaction was stopped with 1N H₂SO₄, and then absorbance was measured. The Ab titer was calculated. The absorbance (OD) of 0.2 was determined as the cut-off value of positive samples.

(Virus Neutralization Titer)

The serum of immunized mice was mixed with RDE II (Denka-Seiken) and incubated overnight at 37° C. After further incubation for 1 hour at 56° C., serially diluted serum in an MEM medium containing 10 mM of HEPES, 1% penicillin-streptomycin, 0.2% bovine albumin, and 10 μg/mL trypsin, and A/California/7/2009(H1N1) influenza viruses with a final infection titer of 100 TCID₅₀/mL were incubated at 37° C. for 30 minutes. They were then added to Madin-Darby canine kidney (MDCK) cells. After 4 days of incubation at 37° C. with 5% CO₂, the MDCK cells were immobilized with 10% formalin at room temperature for 10 minutes. The cells were stained at room temperature for 30 minutes with naphthol blue black solution (0.5 g of naphthol blue black, 0.5 g of sodium acetate, 45 mL of acetate, and 455 mL of distilled water). The stained cells were thoroughly washed with water and then dried. 0.1 M of NaOH was then added to the cells. The plate was read at 630 nm in a microplate reader. The virus neutralization titer was determined by the maximum dilution rate yielding a higher absorbance than the average of positive control and negative control.

(In Vitro Stimulation of Macrophage)

To prepare macrophages, mice were injected i.p. with 3 mL of 4% (w/v) thioglycolate (SIGMA) solution. After 4 days, macrophages were collected from the abdominal cavity and seeded on a 96-well plate. The macrophages were primed for 15 hours with 50 ng/mL LPS and stimulated with an adjuvant for 8 hours. IL-1β or TNFα in the serum was measured by ELISA.

(Statistical Analysis)

The statistical significance (P<0.05) among groups was determined by Dunnett's multiple comparison test or Student's t-test.

### Example 1: Several Hundred Nanometer HAp has Better Adjuvant Activity than Other Larger HAp or Smaller HAp

This Example investigated whether several hundred nanometer HAp has better adjuvant activity than other larger HAp or smaller HAp. The basic experimental procedure is the same as that disclosed in (Materials and Methods).

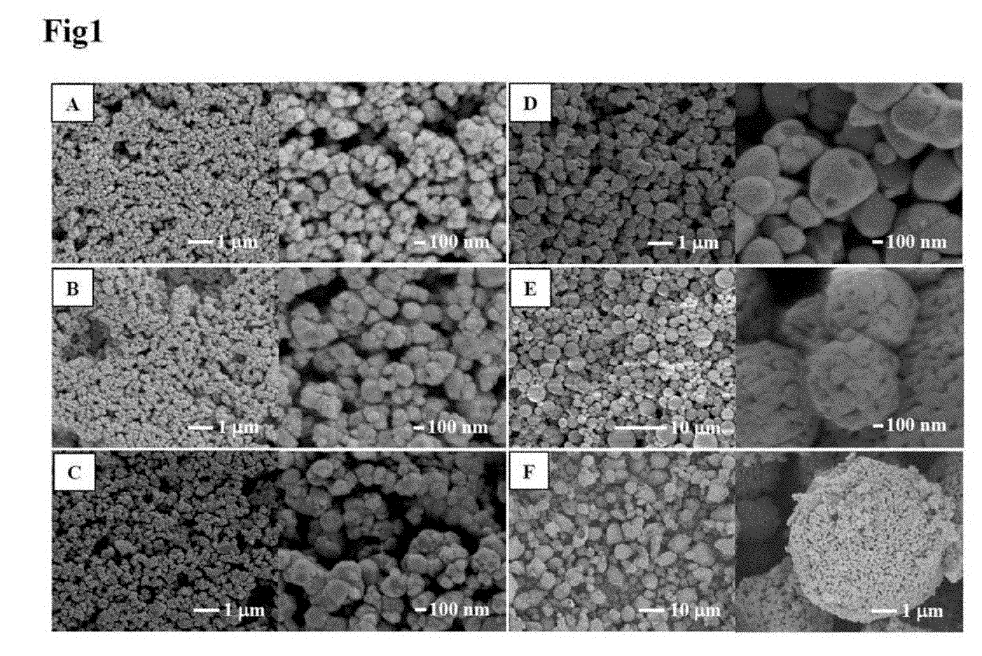

To study the relationship between the HAp particle size and adjuvant activity, the inventors first prepared spherical HAp having a size in the range of nanometers to micrometers including, on average, about 40 nm (S40), about 100 nm (S100), about 170 nm (S170), about 400 nm (S400), about 1.8 μm (S1800), and about 5 μm (S5000) (Table 1 and FIGS. 1A to 1F).

(Table 1)

The HAp sizes were determined by the average value (average particle size) of the length of HAp at 108 points on a picture captured with SEM at 40,000 times magnification. The shape, size, and standard deviation (SD) are shown for each HAp. Thus, the denotation of “size” in Table 1 refers to the “average particle size”.

TABLE 1

| Name | Shape | Size (nm) | SD |
|---|---|---|---|
| S40 | Spherical | 41 | 32 |
| S100 | Spherical | 104 | 74 |
| S170 | Spherical | 172 | 98 |
| S400 | Spherical | 396 | 132 |
| S1800 | Spherical | 1796 | 1093 |
| S5000 | Spherical | 5191 | 3266 |
| R120 | Rod-shaped | 123 | 59 |
| R160 | Rod-shaped | 157 | 82 |
| R250 | Rod-shaped | 253 | 136 |

C57BL/6J mice were immunized twice with the above HAp with an influenza split HA vaccine (SV)+three different doses (0.2 mg, 1 mg, or 5 mg) to test the antigen specific antibody response. About 100 to about 400 nm particles induced a higher antibody response than particles with smaller (about 40 nm) and larger (about 1.8 μm or about 5 μm) size (FIGS. 2A to 2C). IgG subclass analysis revealed that HAp induced greater production of IgG1 than IgG2c, suggesting that HAp induced Th2 polarized immune responses (FIGS. 2B and 2C). Furthermore, immunization with SV+about 100 to about 400 nm particles exhibited a higher virus neutralization titer (FIG. 2D). These results demonstrated that spherical HAp with a size of several hundred nanometers has better adjuvant activity than other larger or smaller HAp.

In view of the above, it was demonstrated that calcium phosphate (hydroxyapatite or the like) with an average particle size greater than 40 nm and less than about 1800 nm, preferably about 100 to about 400 nm, has excellent activity as an immunoadjuvant.

### Example 2: Rod-Shaped HAp Also has Adjuvant Activity

In this Example, the inventors tested the effect of a difference in the shape of hydroxyapatites on immunogenicity or other performances. In this regard, this Example used rod-shaped HAp to compare the difference from spherical HAp studied in Example 1. Other than changing the shape of hydroxyapatites used, the test was conducted as disclosed in (Material and Methods) in accordance with the approach disclosed in Example 1.

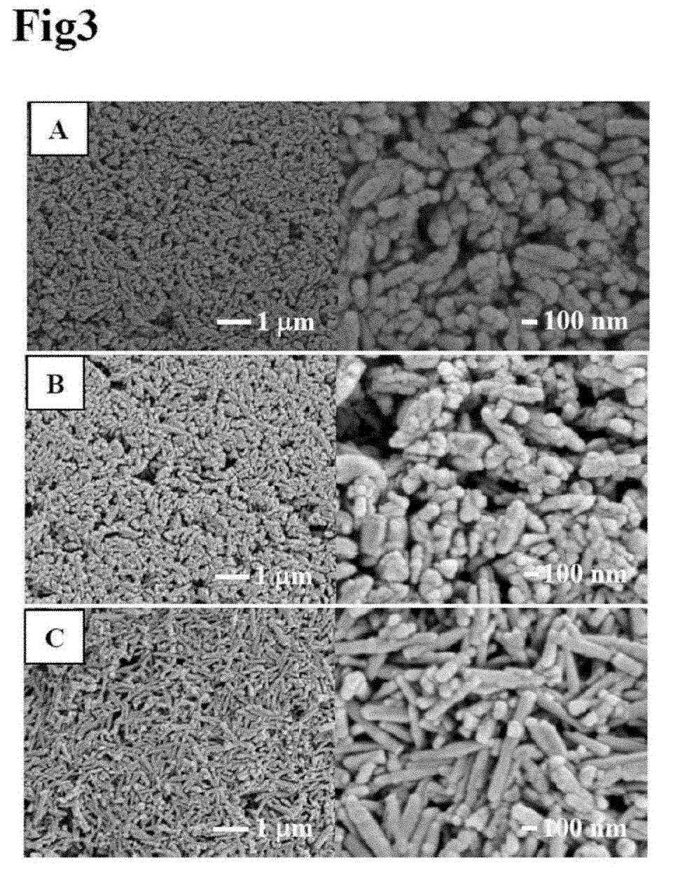

The inventors also prepared rod-shaped HAp, including HAp with a size of about 120 nm (R120), about 160 nm (R160), and about 250 nm (R250) (FIG. 3, Table 1).

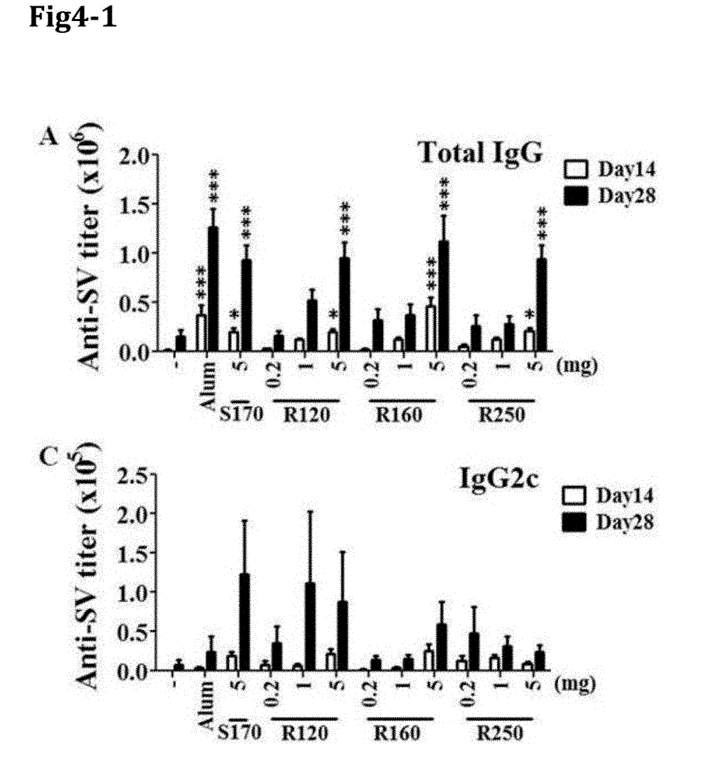

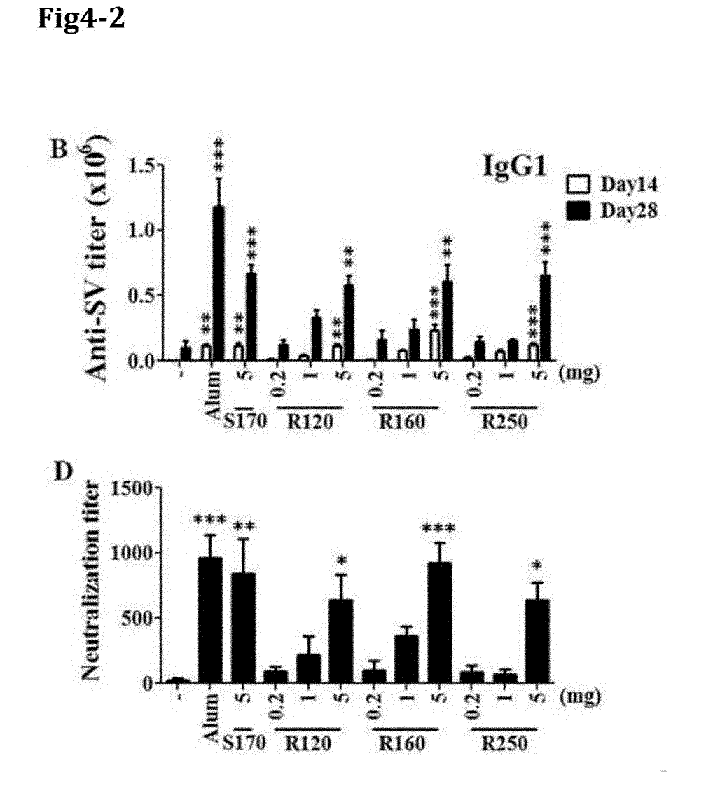

In the same manner as in spherical HAp, C57BL/6J mice were immunized twice with SV+aforementioned rod-shaped HAp to determine antigen specific antibody responses. The rod-shaped HAp induced significantly higher total IgG and IgG1 (FIGS. 4A to 4C) and virus neutralization titer (FIG. 4D) relative to immunization with SV alone, suggesting that rod-shaped HAp also exhibit adjuvant activity and induces Th2 response to SV vaccines. In summary, the optimal size of HAp as a vaccine adjuvant is several hundred nanometers, and spherical and rod-shaped HAps both have similar adjuvant activity to SV vaccines.

Thus, it was discovered that the shape does not affect adjuvant activity as much as the size.

### Example 3: Rod-Shaped HAp Induces Greater Production of IL-β in Microphages

This Example further tested the ability to produce a greater amount of IL-1β in macrophages of rod-shaped hydroxyapatites. This Example was also conducted as disclosed in (Materials and Methods).

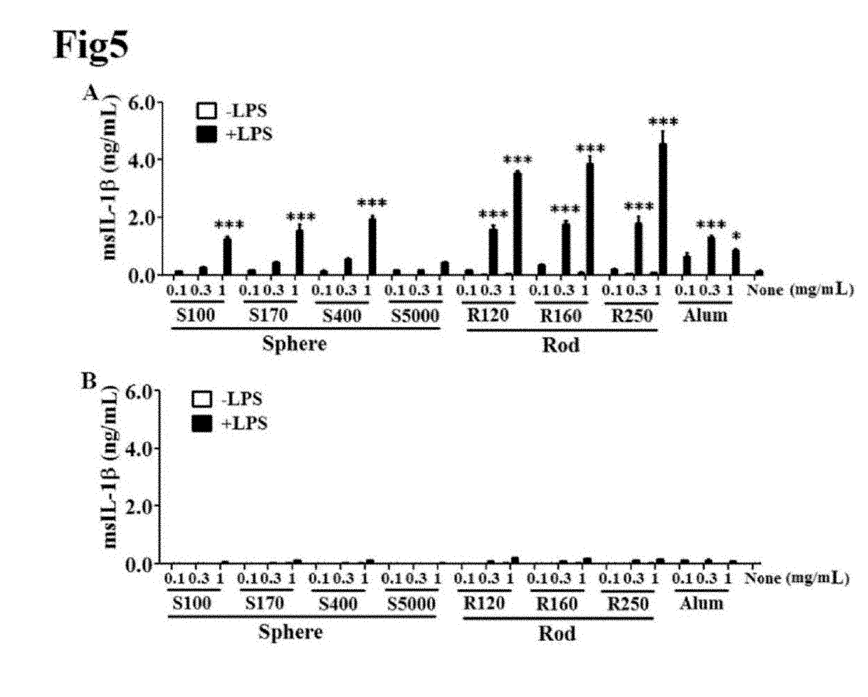

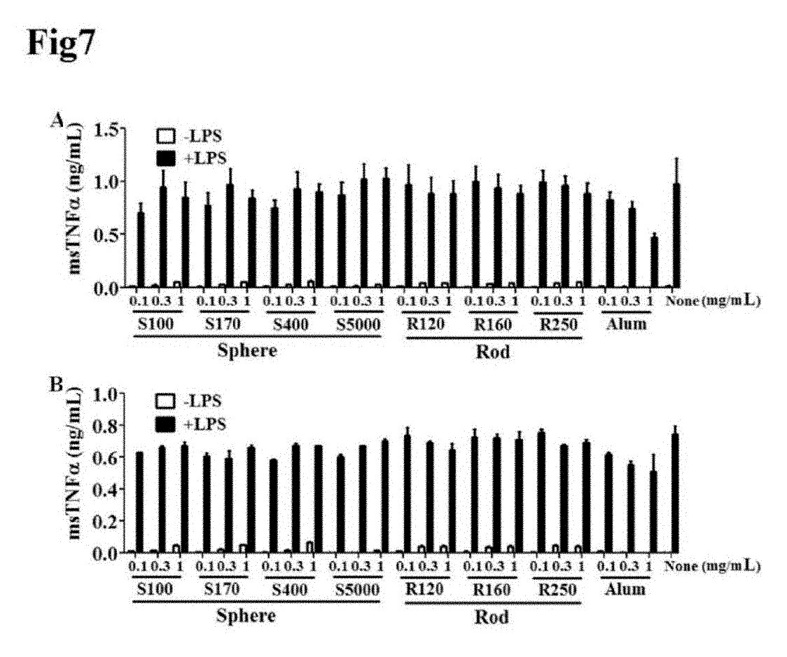

Several reports have demonstrated that particles such as silica or alum stimulate macrophages and dendritic cells to induce production of IL-1β via NLRP3 inflammasome activation. A recent report further demonstrated that rod-shaped or needle-shaped HAp crystals stimulate strong secretion of inflammation promoting cytokines such as IL-1β and IL-18 from macrophages in an NLRP3 inflammasome dependent manner, while spherical HAp crystals has a minimal effect on induction of such cytokines (Jin, C., et al., NLRP3 inflammasome plays a critical role in the pathogenesis of hydroxyapatite-associated arthropathy. Proc Natl Acad Sci USA, 2011. 108(36): p. 14867-72). Thus, to test the ability to produce IL-1β of HAp, thioglycolate inducing peritoneal macrophages were stimulated with spherical or rod-shaped HAp, with lipopolysaccharide (LPS) priming. As reported, rod-shaped HAp induced higher production of IL-1β than spherical HAp (FIG. 5A). Furthermore, larger HAp (S5000) exhibiting weaker adjuvant activity (FIGS. 2-1 and 2-2) did not induce significant IL-1β production. The inventors used peritoneal macrophages from Caspase1−/− mice to further test the involvement of an inflammasome in IL-1β production. Caspase1 is an essential constituent of an inflammasome that cleaves pro-IL-1β to produce its mature form. As observed in alum, the ability to induce IL-1β of HAp is completely dependent on Caspase 1 (FIG. 5B), while TNFα production induced by LPS was not affected by a Caspase1 deletion (FIGS. 7A to B). This shows that HAp induces IL-1β production in an inflammasome dependent manner.

### Example 4: NLRP3 Inflammasome Activation is not Required for Adjuvant Activity of HAp

In the next Example, the inventors conducted a test to demonstrate that NLRP3 inflammasome activation is not required for adjuvant activity of HAp. This Example was also conducted as disclosed in (Materials and Methods).

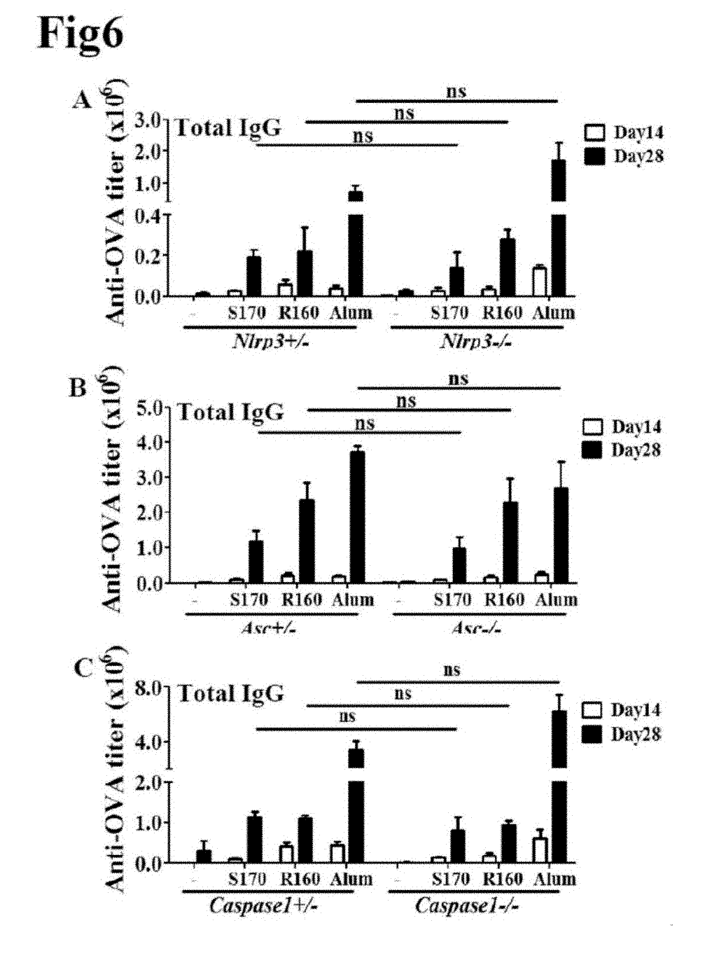

Lastly, the inventors studied the need of NLRP3 inflammasome activation for adjuvant activity of HAp in vivo by immunizing Nlrp3−/− mice, Asc−/− mice or Caspase1−/− mice. For this experiment, the inventors selected 5170 and R160 as representatives of each of spherical and rod-shaped HAp. Nlrp3, Asc, or Caspase1 knockout mice were immunized i.d. twice with OVA-loaded 5170, R160, or alum to test the OVA specific antibody response. All knockout mice tested by the inventors exhibited the same level of OVA specific antibody titer (FIGS. 6A to 6C) despite complete suppression of IL-1β from macrophages in vitro in the absence of Caspase1 (FIGS. 5A and 5B). These results demonstrated that adjuvant activity of HAp is not dependent on in vivo NLRP3 inflammasome activation.

### DISCUSSION

Adjuvants that cannot be metabolized such as alum may be retained for a long period of time to induce hardening at the site of injection. Thus, adjuvants that cannot be metabolized have a potential safety and reactogenic problem at the site of injection. HAp, which is the main constituent of bones or teeth, is expected to be a biocompatible material. In fact, it is shown that HAp is solubilized in acidic or basic medium (JARCHO, M., Calcium Phosphate Ceramics as Hard Tissue Prosthetics.) and decomposed by macrophages.

The particle size is important in adjuvant activity (Oyewumi, M. O., A. Kumar, and Z. Cui, Nano-microparticles as immune adjuvants: correlating particle sizes and the resultant immune responses. Expert Rev Vaccines, 2010. 9(9): p. 1095-107., Yan, S., W. Gu, and Z. P. Xu, Re-considering how particle size and other properties of antigen-adjuvant complexes impact on the immune responses. J Colloid Interface Sci, 2013. 395: p. 1-10). For example, bovine serum albumin (BSA) loaded poly(d,l-lactic-co-glycolic acid) (PLGA) microparticles or polylactic acid (PLA) microparticles loaded with a hepatitis B surface antigen induce a higher serum IgG antibody response than nanoparticles (Gutierro., I., et al., Size dependent immune response after subcutaneous, oral and intranasal administration of BSA loaded nanospheres. Vaccine, 2002. 21: p. 67-77., Kanchan, V. and A. K. Panda, Interactions of antigen-loaded polylactide particles with macrophages and their correlation with the immune response. Biomaterials, 2007. 28(35): p. 5344-5357). On the other hand, tetanus toxoid-loaded sulfobutylated poly(vinyl alcohol) graft PLGA nanoparticles induce a higher antibody response than microparticles (Jung, T., et al., Tetanus toxoid loaded nanoparticles from sulfobutylated poly(vinyl alcohol)-graft-poly(lactide-co-glycolide): evaluation of antibody response after oral and nasal application in mice. Pharm Res, 2001. 18(3): p. 352-60). About 40 nm induced the strongest ovalbumin (OVA) specific antibody response in a comparison of immune responses induced by OVA conjugated polystyrene beads of different sizes (about 20 nm, about 40 nm, about 100 nm, about 500 nm, about 1 μm, and about 2 μm) (Fifis, T., et al., Size-Dependent Immunogenicity: Therapeutic and Protective Properties of Nano-Vaccines against Tumors. The Journal of Immunology, 2004. 173(5): p. 3148-3154). These reports suggest the presence of an optimal size range for each particle as an adjuvant. The adjuvant activity was compared for HAp with different sizes from about 40 nm to about 5 μm in a study of the inventors (FIGS. 2-1 and 2-2). The findings of the inventors demonstrated that about 100 to about 400 nm is the optimal size range of HAp for adjuvant activity. However, the shape was not essential for adjuvant activity (FIGS. 4-1 and 4-2).

The action mechanism of particulate adjuvants is understood to be a depot effect, incorporation of an antigen into antigen presenting cells, and activation of innate immunity. Some reports have demonstrated that NLRP3 inflammasome activation is essential for adjuvant activity of alum (Eisenbarth, S.C., et al., Crucial role for the Nalp3 inflammasome in the immunostimulatory properties of aluminium adjuvants. Nature, 2008. 453(7198): p. 1122-6., Li, H., et al., Cutting Edge: Inflammasome Activation by Alum and Alum's Adjuvant Effect Are Mediated by NLRP3. The Journal of Immunology, 2008. 181(1): p. 17-21). It is understood that NLRP3 inflammasome activity is required for adjuvant activity because other particular adjuvants also require NLRP3 inflammasome activation in vitro. However, other reports show that adjuvant activity of particles including alum is non-NLRP3 inflammasome dependent (Coban, C., et al., Immunogenicity of whole-parasite vaccines against Plasmodium falciparum involves malarial hemozoin and host TLR9. Cell Host Microbe, 2010. 7(1): p. 50-61., McKee, A. S., et al., Alum induces innate immune responses through macrophage and mast cell sensors, but these sensors are not required for alum to act as an adjuvant for specific immunity. J Immunol, 2009. 183(7): p. 4403-14., Franchi, L. and G. Nunez, The Nlrp3 inflammasome is critical for aluminium hydroxide-mediated IL-1beta secretion but dispensable for adjuvant activity. Eur J Immunol, 2008. 38(8): p. 2085-9). Thus, the inventors tested the involvement of NLRP3 inflammasomes in adjuvant activity of HAp. Even if the production of inflammation promoting cytokine IL-113 induced by HAp was completely suppressed in the absence of Caspase1 in vitro, a significant antibody response was induced in the absence of an NLRP3 inflammasome constituent Nlrp3, Asc, or Caspase1 in vivo (FIGS. 5 and 6). It is noteworthy that adjuvant activity of alum was not affected by the absence of Nlrp3, Asc, or Caspase1 in this Example. These results revealed that NLRP3 inflammasome activation is not required for adjuvant activity of particles, both HAp and alum.

In summary, the results of this Example showed that the range of sizes affects the adjuvant activity of particles in vivo, and is a major controlling factor. Innate immunity signaling is generally essential for induction of an adaptive immune response, but the results of this Example show that NLRP3 inflammasome dependent IL-113 production is not an absolute condition for particulate adjuvants such as alum and HAp. These findings have a significant meaning for designing the development of additional particulate adjuvants.

### LIST OF MENTIONED REFERENCES

The documents in the list are not provided for the purpose of acknowledging the documents as prior art to the present invention)
1. FURUHASHI., K., et al., Evaluation of Adhesion between Material and Epithelium using a Three-dimensional Human Epidermal Model. Nano Biomedicine, 2012. 4(2): p. 76-84.
2. HATAKEYAMA., W., et al., Bone-regeneration Trial of Rat Critical-size Calvarial Defects using Nano-apatite/collagen Composites. Nano Biomedicine, 2013. 5(2): p. 95-103.
3. JARCHO, M., Calcium Phosphate Ceramics as Hard Tissue Prosthetics.
4. Kwong C. H., et al., Solubilization of hydroqxapatite crystals by murine bone cells, macrophages and fibroblasts. Biomaterials., 1989. 10: p. 579-84.
5. Tritto, E., F. Mosca, and E. De Gregorio, Mechanism of action of licensed vaccine adjuvants. Vaccine, 2009. 27(25-26): p. 3331-4.
6. Brito, L. A., P. Malyala, and D. T. O'Hagan, Vaccine adjuvant formulations: a pharmaceutical perspective. Semin Immunol, 2013. 25(2): p. 130-45.
7. Hedayat, M., K. Takeda, and N. Rezaei, Prophylactic and therapeutic implications of toll-like receptor ligands. Med Res Rev, 2012. 32(2): p. 294-325.
8. TCHAVDAR L, V., Aluminium Phosphate but Not Calcium Phosphate Stimulates the Specific IgE Response in Guinea Pigs to Tetanus Toxoid. Allergy, 1978. 33: p. 155-9.
9. Aggerbeck, H. and I. Heron, Adjuvanticity of aluminium hydroxide and calcium phosphate in diphtheria-tetanus vaccines-I. Vaccine, 1995. 13(14): p. 1360-5.
10. Aggerbeck, H., C. Fenger, and I. Heron, Booster vaccination against diphtheria and tetanus in man. Comparison of calcium phosphate and aluminium hydroxide as adjuvants-II. Vaccine, 1995. 13(14): p. 1366-74.
11. HE, Q., et al., Calcium Phosphate Nanoparticle Adjuvant. CLINICAL AND DIAGNOSTIC LABORATORY IMMUNOLOGY, 2000. 7(6): p. 899.
12. Kuroda, E., C. Coban, and K. J. Ishii, Particulate Adjuvant and Innate Immunity: Past Achievements, Present Findings, and Future Prospects. International Reviews of Immunology, 2013. 32(2): p. 209-220.
13. Jiang, D., et al., Structure and adsorption properties of commercial calcium phosphate adjuvant. Vaccine, 2004. 23(5): p. 693-8.
14. Jin, C., et al., NLRP3 inflammasome plays a critical role in the pathogenesis of hydroxyapatite-associated arthropathy. Proc Natl Acad Sci USA, 2011. 108(36): p. 14867-72.
15. He, Q., et al., Calcium Phosphate Nanoparticles Induce Mucosal Immunity and Protection against Herpes Simplex Virus Type 2. Clinical and Vaccine Immunology, 2002. 9(5): p. 1021-1024.
16. Oyewumi, M. O., A. Kumar, and Z. Cui, Nano-microparticles as immune adjuvants: correlating particle sizes and the resultant immune responses. Expert Rev Vaccines, 2010. 9(9): p. 1095-107.
17. Yan, S., W. Gu, and Z. P. Xu, Re-considering how particle size and other properties of antigen-adjuvant complexes impact on the immune responses. J Colloid Interface Sci, 2013. 395: p. 1-10.
18. Gutierro., I., et al., Size dependent immune response after subcutaneous, oral and intranasal administration of BSA loaded nanospheres. Vaccine, 2002. 21: p. 67-77.
19. Kanchan, V. and A. K. Panda, Interactions of antigen-loaded polylactide particles with macrophages and their correlation with the immune response. Biomaterials, 2007. 28(35): p. 5344-5357.
20. Jung, T., et al., Tetanus toxoid loaded nanoparticles from sulfobutylated poly(vinyl alcohol)-graft-poly(lactide-co-glycolide): evaluation of antibody response after oral and nasal application in mice. Pharm Res, 2001. 18(3): p. 352-60.
21. Fifis, T., et al., Size-Dependent Immunogenicity: Therapeutic and Protective Properties of Nano-Vaccines against Tumors. The Journal of Immunology, 2004. 173(5): p. 3148-3154.
22. Eisenbarth, S. C., et al., Crucial role for the Nalp3 inflammasome in the immunostimulatory properties of aluminium adjuvants. Nature, 2008. 453(7198): p. 1122-6.
23. Li, H., et al., Cutting Edge: Inflammasome Activation by Alum and Alum's Adjuvant Effect Are Mediated by NLRP3. The Journal of Immunology, 2008. 181(1): p. 17-21.
24. Coban, C., et al., Immunogenicity of whole-parasite vaccines against Plasmodium falciparum involves malarial hemozoin and host TLR9. Cell Host Microbe, 2010. 7(1): p. 50-61.
25. McKee, A. S., et al., Alum induces innate immune responses through macrophage and mast cell sensors, but these sensors are not required for alum to act as an adjuvant for specific immunity. J Immunol, 2009. 183(7): p. 4403-14.
26. Franchi, L. and G. Nunez, The Nlrp3 inflammasome is critical for aluminium hydroxide-mediated IL-1beta secretion but dispensable for adjuvant activity. Eur J Immunol, 2008. 38(8): p. 2085-9.
27. Onishi, M., et al., Hydroxypropyl-beta-Cyclodextrin Spikes Local Inflammation That Induces Th2 Cell and T Follicular Helper Cell Responses to the Coadministered Antigen. J Immunol, 2015.
28. Okada, M. and T. Furuzono, Nano-Sized Ceramic Particles of Hydroxyapatite Calcined with an Anti-Sintering Agent. Journal of Nanoscience and Nanotechnology, 2007. 7(3): p. 848-851.
29. Okada, M. and T. Furuzono, Calcination of Rod-like Hydroxyapatite Nanocrystals with an Anti-sintering Agent Surrounding the Crystals. Journal of Nanoparticle Research, 2006. 9(5): p. 807-815.

As disclosed above, the present invention is exemplified by the use of its preferred embodiments. However, it is understood that the scope of the present invention should be interpreted solely based on the Claims. It is also understood that any patent, any patent application, and any references cited herein should be incorporated herein by reference in the same manner as the contents are specifically described herein. The present application claims priority to Japanese Patent Application No. 2015-183631 filed on Sep. 17, 2015 filed in Japan. The entire content thereof is incorporated herein by reference.

### INDUSTRIAL APPLICABILITY

Industrial applicability of the present invention is found in the pharmaceutical industry.

1. (canceled)

2. The method of claim 12, wherein the average particle size of the calcium phosphate is about 100 to about 400 nm.

3. The method of claim 12, wherein the calcium phosphate is a hydroxyapatite.

4. The method of claim 12, wherein the calcium phosphate is rod-shaped.

5. (canceled)

6. The method of claim 12, wherein NLRP3 inflammasome activation is not required.

7. The method of claim 12, wherein a Th2 response is enhanced.

8. (canceled)

9. The method of claim 12, further comprising the step of administering a vaccine.

10. (canceled)

11. (canceled)

12. A method of preventing or treating a disease requiring a vaccine comprising an immunoadjuvant comprising calcium phosphate with an average particle size greater than about 40 nm and less than about 1800 nm, comprising administering to a subject an effective amount of the vaccine.

13. A method of preventing or treating a disease requiring a vaccine comprising an immunoadjuvant comprising rod-shaped calcium phosphate, comprising administering to a subject an effective amount of the vaccine.

14. The method of claim 13, wherein NLRP3 inflammasome activation is not required.

15. The method of claim 13, wherein a Th2 response is enhanced.

16. The method according to claim 13, further comprising the step of administering a vaccine.
